(19) United States
(12) Patent Application Publication
(10) Pub. No.: US 2010/0273976 A1
(43) Pub. Date: Oct. 28, 2010

(54) POLYIMIDE-BASED MATERIAL, COMPOSITION AND FILM, AND MANUFACTURING METHOD THEREOF

(75) Inventors: Rozhanskii IGOR, Tokyo (JP); Takaaki Uno, Tokyo (JP); Takashi Okada, Tokyo (JP); Kohei Goto, Tokyo (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: JSR CORPORATION, Tokyo (JP)

(21) Appl. No.: 12/646,494

(22) Filed: Dec. 23, 2009

(30) Foreign Application Priority Data
Dec. 26, 2008 (JP) 2008-335339

(51) Int. Cl. C08G 73/10; B05D 3/02; C07C 61/06; C08G 73/00

(52) U.S. Cl. 528/322; 427/385.5; 562/503; 528/363

(57) ABSTRACT

A polyimide-type material that exhibits an excellent heat resistance and moldability such as easiness of molding into a film configuration and low process load, and a film made from this polyimide-type material are provided. The polyimide-type material contains a polyamic acid and/or polyimide obtained by reacting (A) at least one acyl compound selected from the group consisting of 1-cis-2-cis-3-trans-4-trans-cyclopentanetetracarboxylic acid, 1-cis-2-cis-3-trans-4-trans-cyclopentanetetracarboxylic acid dianhydride, and reactive derivatives thereof, with (B) an imino-forming compound.

### BACKGROUND OF THE INVENTION

1. Field of the Invention

The present invention relates to novel acyl compounds, a polyimide-type material including a polyamic acid and/or polyimide, which is prepared by using the acyl compounds, a composition including this polyimide-type material, a film formed of this polyimide-type material, and a method of producing this film.

2. Description of the Related Art

Fully aromatic polyimides, which are prepared by reacting an aromatic tetracarboxylic acid dianhydride with an aromatic diamine, generally exhibit an excellent heat resistance, excellent mechanical properties, excellent electrical properties, and an excellent resistance to oxidation and hydrolysis, which arise from the molecular rigidity, resonance stabilization of the molecule, and strong chemical bonds therein. As a result, fully aromatic polyimides are in wide use as films, coatings, molded articles, and dielectric materials in fields such as the electrical industry, battery industry, automotive industry, and aerospace industry.

However, fully aromatic polyimide films, as typified by, for example, Kapton (trade name and registered trademark, Toray-Dupont Co., Ltd.) have the problem of a poor moldability due to the high process load for molding into films such as the necessity of thermal processing at high temperatures, and also have the problem of limitation on the use of these films as optical materials. In specific terms, the polyimides that form such films exhibit a low solubility in organic solvents. As a consequence, the polyimide film must be obtained by the following procedures. A solution in which the polyamic acid precursor for the polyimide is dissolved in organic solvent is used. A film-like coating layer is formed by the application of this solution to a substrate. After that, the coating layer is heated to a high temperature of about 400° C. in order to imidize the polyamic acid in the coating layer and provide the polyimide film.

On the other hand, semi-aromatic polyimides provided by reacting an alicyclic tetracarboxylic acid dianhydride with an aromatic diamine have recently been reported. For example, a polyimide obtained by reacting cyclobutanetetracarboxylic acid dianhydride and 1,2,4,5-cyclohexanetetracarboxylic acid dianhydride with an aromatic diamine such as 4,4′-diaminodiphenyl ether has been proposed (High Performance Polymers, Vol. 19, pp. 175-193 (2007)).

A polyimide resin that is obtained by reacting at least one acyl-containing compound selected from the group consisting of 1,2,4,5-cyclohexanetetracarboxylic acid, 1,2,4,5-cyclohexanetetracarboxylic acid dianhydride and their reactive derivatives, with at least one imino-forming compound selected from compounds represented by a specific formula and having at least one phenylene group and isopropylidene group has also been proposed (Japanese Patent Application Laid-open No. 2006-199945).

### SUMMARY OF THE INVENTION

The polyimide described in High Performance Polymers, Vol. 19, pp. 175-193 (2007) has an excellent heat resistance, but still exhibits a low solubility in organic solvents and as a consequence requires thermal processing (i.e. thermal imidization) at a high temperature using the polyamic acid precursor when the polyimide is formed into a film. Thus, the problem of a large process load and hence a poor moldability still remains.

The polyimide resin described in Japanese Patent Application Laid-open No. 2006-199945 exhibits an unsatisfactory heat resistance and as a consequence cannot be used as an optical element.

Accordingly, the purpose of the present invention is to introduce novel acyl compounds that can provide a polyimide-type material that exhibits an excellent heat resistance and an excellent moldability (i.e. easiness of molding into a film, and low process load). Additional objects of the present invention is to produce a polyimide-type material, a composition including this polyimide-type material, a film made from this polyimide-type material, and a method of producing this film.

As a result of intensive investigations in order to solve the problems described above, the present inventor succeeded in synthesizing novel acyl compounds and discovered that the aforementioned objects of the present invention could be achieved by a polyimide-type material that includes a polyamic acid and/or polyimide obtained by reacting these acyl compounds with an imino-forming compound (i.e. diamine, diisocyanate, or trialkylsilylated diamine). The present invention was achieved based on this discovery.

That is, the present invention provides the following [1] to [10].

[1] A polyimide-type material comprising at least one selected from polyamic acids and polyimides obtained by reacting

(A) at least one acyl compound selected from the group consisting of 1-cis-2-cis-3-trans-4-trans-cyclopentanetetracarboxylic acid, 1-cis-2-cis-3-trans-4-trans-cyclopentanetetracarboxylic acid dianhydride, and reactive derivatives thereof, with

(B) an imino-forming compound.

[2] The polyimide-type material according to [1] above, wherein the imino-forming compound (B) is a diamine compound.

[3] A polyimide-type resin composition comprising a polyimide-type material according to the preceding [1] or [2] and an organic solvent.

[4] A film comprising the polyimide-type material according to the preceding [1] or [2].

[5] The film according to [4] above, used for an optical element.

[6] The film according to [4] above, used for a printed wiring substrate (i.e. a printed circuit board).

[7] A method of producing the film according to any one of the preceding [4] to [6], comprising the steps of:

forming a coating layer by coating a substrate with a solution comprising organic solvent and a polyamic acid and/or polyimide obtained by reacting the aforementioned acyl compound (A) with the aforementioned imino-forming compound (B); and

obtaining a film by removing the solvent from the coating layer by evaporation.

[8] 1-cis-2-cis-3-trans-4-trans-cyclopentanetetracarboxylic acid.

[9] 1-cis-2-cis-3-trans-4-trans-cyclopentanetetracarboxylic acid dianhydride.

[10] A material for synthesizing a polyamic acid and/or a polyimide, comprising 1-cis-2-cis-3-trans-4-trans-cyclopentanetetracarboxylic acid and/or 1-cis-2-cis-3-trans-4-trans-cyclopentanetetracarboxylic acid dianhydride.

The polyimide-type material of the present invention, which comprises a polyamic acid and/or a polyimide (also referred to below as “polyimide and so forth”) exhibits an excellent heat resistance and an excellent solubility in organic solvents, because the polyimide-type material is provided by reacting a specific acyl compound with an imino-forming compound.

Thus, the polyimide-type material of the present invention, which has an excellent solubility in organic solvents, enables film formation to be carried out through the use of a solution provided by the direct dissolution of this polyimide-type material in an organic solvent. In this case, a coating layer is formed by coating a substrate with a solution containing the aforementioned polyimide and so forth, and after that, heating is performed at a temperature sufficient to evaporate the solvent in the coating layer. This achieves a reduction in the process load, because for example, it is no longer necessary to perform heating at a high temperature above 400° C. as in the case of thermal imidization using a solution containing polyamic acid and organic solvent.

In addition, a film having a high glass-transition temperature and thus an excellent heat resistance can be obtained using the polyimide-type material of the present invention.

### BRIEF DESCRIPTION OF THE DRAWINGS

FIG. 1 is a chart that shows the NMR spectrum of the compound obtained in Example 1.

FIG. 2 is a chart that shows the NMR spectrum (¹H, DMSO-d) of the compound obtained in Example 2.

FIG. 3 is a chart that shows the NMR spectrum (³¹C, CDCl₁) of the compound obtained in Example 2.

FIG. 4 is a chart that shows the IR spectrum of the polymer obtained in Example 3.

FIG. 5 is a chart that shows the IR spectrum of the polymer obtained in Example 4.

FIG. 6 is a chart that shows the IR spectrum of the polymer obtained in Comparative Example 1.

FIG. 7 is a chart that shows the IR spectrum of the polymer obtained in Comparative Example 2.

FIG. 8 is a chart that shows the IR spectrum of the polymer obtained in Comparative Example 3.

FIG. 9 is a chart that shows the IR spectrum of the polymer obtained in Comparative Example 4.

### DESCRIPTION OF THE PREFERRED EMBODIMENTS OF THE INVENTION

The main constituent of the polyimide-type material of the present invention is polyamic acid and/or polyimide provided by the reaction of a novel acyl compound (referred to as component (A)) with an imino-forming compound (referred to as component (B)).

The novel acyl compounds of the present invention will be described first.

[The acyl compounds; component (A)]

The novel acyl compounds of the present invention are at least one acyl compound selected from the group consisting of 1-cis-2-cis-3-trans-4-trans-cyclopentanetetracarboxylic acid, 1-cis-2-cis-3-trans-4-trans-cyclopentanetetracarboxylic acid dianhydride, and their reactive derivatives.

The 1-cis-2-cis-3-trans-4-trans-cyclopentanetetracarboxylic acid can be exemplified by the compound given by formula (1-1) below or the compound given by formula (1-2) below. The 1-cis-2-cis-3-trans-4-trans-cyclopentanetetracarboxylic acid dianhydride can be exemplified by the compound given by formula (2-1) below or the compound given by formula (2-2) below.

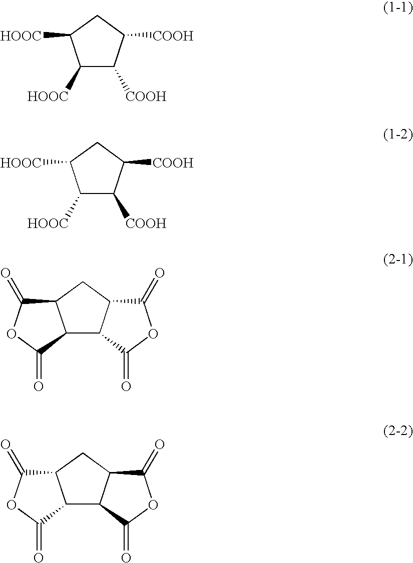

The reactive derivatives are compounds that can convert to 1-cis-2-cis-3-trans-4-trans-cyclopentanetetracarboxylic acid. Suitably used in this regard are, for example, ester compounds in which one or two of the carboxyl groups in the 1-cis-2-cis-3-trans-4-trans-cyclopentanetetracarboxylic acid are esterified, and acid chlorides in which one or two of the carboxyl groups have been converted into the acid chloride.

The aforementioned ester-type reactive derivatives can be exemplified by alkyl esters such as the monomethyl ester of 1-cis-2-cis-3-trans-4-trans-cyclopentanetetracarboxylic acid, the dimethyl ester of 1-cis-2-cis-3-trans-4-trans-cyclopentanetetracarboxylic acid, the trimethyl ester of 1-cis-2-cis-3-trans-4-trans-cyclopentanetetracarboxylic acid, the tetramethyl ester of 1-cis-2-cis-3-trans-4-trans-cyclopentanetetracarboxylic acid, the monoethyl ester of 1-cis-2-cis-3-trans-4-trans-cyclopentanetetracarboxylic acid, the diethyl ester of 1-cis-2-cis-3-trans-4-trans-cyclopentanetetracarboxylic acid, the triethyl ester of 1-cis-2-cis-3-trans-4-trans-cyclopentanetetracarboxylic acid, the tetraethyl ester of 1-cis-2-cis-3-trans-4-trans-cyclopentanetetracarboxylic acid and so forth; and aryl esters in which the preceding alkyl esters have been replaced by unsubstituted phenyl esters or substituted phenyl esters such as para-substituted phenyl esters and so forth.

The acid chloride-type reactive derivatives can be exemplified by the tetrachloride of 1-cis-2-cis-3-trans-4-trans-cyclopentanetetracarboxylic acid and the dichloride of an ester of 1-cis-2-cis-3-trans-4-trans-cyclopentanetetracarboxylic acid (wherein the alcohol or phenol moiety in the ester is the same as those described above).

The acyl compounds described above are well suited for use as materials for the synthesis of polyimide and so forth. A polyimide and so forth that exhibits an excellent heat resistance can be obtained in this case.

A single one or a combination of two or more of the previously described acyl compounds can be used to synthesize the polyimide and so forth.

The compound represented by formula (1-1) or (1-2) described above (i.e. 1-cis-2-cis-3-trans-4-trans-cyclopentanetetracarboxylic acid) and the compound represented by formula (2-1) or (2-2) described above (i.e. 1-cis-2-cis-3-trans-4-trans-cyclopentanetetracarboxylic acid dianhydride) are preferred for the acyl compounds of the present invention.

The compound represented by formula (2-1) or (2-2) above is more preferred. The use of 1-cis-2-cis-3-trans-4-trans-cyclopentanetetracarboxylic acid dianhydride, which is an anhydride, makes possible polyamic acid synthesis at a lower temperature than for the use of the non-anhydride.

With reference to these acyl compounds, the compound represented by formula (1-1) or (1-2) above can be produced, for example, by heating all-cis 1,2,3,4-cyclopentanetetracarboxylic acid. The heating temperature is generally 200 to 320° C. and is preferably 250 to 300° C. The heating time is generally 0.1 to 10 hours and is preferably 2 to 8 hours. While the atmosphere during heating is not particularly restricted, heating is preferably carried out in air or an inert gas such as nitrogen, argon, helium and so forth, and is more preferably carried out in an inert gas such as nitrogen, argon, helium and so forth.

The compound represented by formula (2-1) or (2-2) above can be produced, for example, by adding a dehydrating agent such as acetic anhydride, acetyl chloride and so forth to 1-cis-2-cis-3-trans-4-trans-cyclopentanetetracarboxylic acid and heating.

The ester compound and the acid chloride of the compound represented by formula (1-1) or (1-2) above can be produced, for example, by reacting 1-cis-2-cis-3-trans-4-trans-cyclopentanetetracarboxylic acid with an alcohol, thionyl chloride, phosphorus trichloride and so forth.

The imino-forming compound (referred to as component (B)), which reacts with the above-described acyl compound to provide the polyimide-type material of the present invention, is described in the following. An imino-forming compound denotes a compound that will form the imino group (i.e. —NH— structure) when being reacted with component (A) (i.e. acyl compound).

### [Component (B)]

The imino-forming compound (B) can be exemplified by diamine compounds, diisocyanate compounds, and disilylamine compounds. Examples of such compounds include compounds represented by the following formula (3).

X-Q-X (3)

(In formula (3), X is —NH, —N═C═O, or —NHSi(R₁) (R₂) (R₃); Q represents a divalent organic group; and R₁ to R₃ each independently represent an alkyl group having 1 to 15 carbon atoms.)

This imino-forming compound can be specifically exemplified by the following: aromatic diamines such as p-phenylendiamine, m-phenylenediamine, 4,4′-diaminodiphenylmethane, 4,4′-diaminodiphenylethane, 4,4′-diaminodiphenyl sulfide, 4,4′-diaminodiphenyl sulfone, 4,4′-diaminodiphenyl ether, 1,5-diaminonaphthalene, 3,3′-dimethyl-4,4′-diaminobiphenyl, 4,4′-diaminobenzanilide, diaminodiphenyl ether, 3,3′-diaminobenzophenone, 3,4′-diaminobenzophenone, 4,4′-diaminobenzophenone, 2,2-bis[4-(4-aminophenoxy)phenyl]propane, 2,2-bis[4-(4-aminophenoxy)phenyl]hexafluoropropane, 2,2-bis(4-aminophenyl)hexafluoropropane, bis[4-(4-aminophenoxy)phenyl]sulfone, 1,4-bis(4-aminophenoxy)benzene, 4,4′-(p-phenylenediisopropylidene)bisaniline, 4,4′-(m-phenylenediisopropylidene)bisaniline, 1,3-bis(4-aminophenoxy)benzene, 1,3-bis(3-aminophenoxy)benzene, 9,9-bis(4-aminophenyl)-10-hydroanthracene, 9,9-bis(4-aminophenyl)fluorene, 4,4′-methylene-bis(2-chloroaniline), 2,2′,5,5′-tetrachloro-4,4′-diaminobiphenyl, 2,2′-dichloro-4,4′-diamino-5,5′-dimethoxybiphenyl, 3,3′-dimethoxy-4,4′-diaminobiphenyl, and so forth; aromatic diamines that contain a heteroatom such as diaminotetraphenylthiophene and so forth; aliphatic or alicyclic diamines such as 1,1-metaxylylenediamine, 1,2-ethylenediamine, 1,3-propanediamine, tetramethylenediamine, pentamethylenediamine, hexamethylenediamine, heptamethylenediamine, octamethylenediamine, nonamethylenediamine, 1,4-diaminocyclohexane, isophoronediamine, tetrahydrodicyclopentadienylenediamine, hexahydro-4,7-methanoindanylenedimethylenediamine, tricyclo[6.2.1.0]undecylenedimethyldiamine, 4,4′-methylenebis(cyclohexylamine), and so forth. A single one of these may be used, or two or more of these may be used in combination. Commercially available products can be directly used for these diamine compounds, or the commercial products can be used after re-reduction for these diamine compounds.

A method of producing the polyimide-type material of the present invention and the method of producing a film according to the present invention are described in the following.

The polyimide-type material of the present invention can be produced by a method comprising the steps of: (a) reacting the aforementioned component (A) with the aforementioned component (B) to produce a solution including a polyamic acid and organic solvent; and (b) imidizing at least a portion (i.e. a part) of the polyamic acid.

The method of producing a film according to the present invention comprises the steps of: forming a coating layer by coating a supporting substrate (i.e. a supporting board) with a solution including an organic solvent and a polyamic acid and/or polyimide obtained by reacting the aforementioned component (A) with the aforementioned component (B); and obtaining a film by removing the organic solvent from the coating layer by evaporation.

The method of producing a film according to the present invention can include a preliminary step of preparing a solution including an organic solvent and a polyamic acid and/or polyimide. In this case, the method of producing a film according to the present invention includes the steps of: preparing a solution including an organic solvent and a polyamic acid and/or polyimide by reacting the aforementioned component (A) with and the aforementioned component (B) (for example, the previously described steps (a) and (b)); forming a coating layer by coating a supporting substrate with the solution including an organic solvent and a polyamic acid and/or polyimide (referred to as step (c)); and obtaining a film by removing the organic solvent from the coating layer by evaporation (referred to as step (d)).

### [Step (a) ]

Step (a) is a step of reacting the aforementioned component (A) with the aforementioned component (B) to produce a solution including a polyamic acid and an organic solvent.

An example of step (a) is a procedure in which at least one imino-forming compound (B) is dissolved in an organic solvent; after that, at least one acyl compound (A) is added to the resulting solution; and stirring is then performed for 1 to 60 hours at a temperature of 0 to 100° C. Another example of step (a) is a procedure in which at least one acyl compound (A) is dissolved in an organic solvent; after that, at least one imino-forming compound (B) is added to the resulting solution; and stirring is then performed for 1 to 60 hours at a temperature of 0 to 100° C.

The organic solvent described above can be exemplified by polar aprotic solvents such as N-methyl-2-pyrrolidone, N,N-dimethylacetamide, N,N-dimethylformamide, dimethyl sulfoxide, γ-butyrolactone, N,N′-dimethylimidazolidinone, tetramethylurea and so forth, and phenolic solvents such as cresol, xylenol, halogenated phenol and so forth. N-methyl-2-pyrrolidone and N,N-dimethylacetamide are preferred among the preceding.

A single one or a mixture of two or more of these organic solvents may be used.

The sum of the amounts of imino-forming compound (B) and acyl compound (A) in the reaction solution is preferably 5 to 30 mass % of the total amount of the reaction solution.

The ratio between the acyl compound (A) and the imino-forming compound (B) is preferably a ratio that provides 0.8 to 1.2 equivalents and more preferably 1.0 to 1.1 equivalents of component (A) per 1 equivalent of component (B). A value less than 0.8 equivalent or more than 1.2 equivalents results in a low molecular weight that can make film formation difficult.

The terminals of the polyamic acid and/or polyimide obtained in the aforementioned method take the form of carboxylic acid anhydride or amine. These polymer terminal groups enable film formation to occur as is and without further treatment. Also, the terminal carboxylic acid anhydride groups can be treated by the addition of a monofunctional aromatic amine as typified by aniline derivatives.

The term of “polyamic acid” means an acid that has a structure that is produced by the reaction of an acid anhydride group with an amino group and that contains —CO—NH— and —CO—OH, or means the derivative of such an acid (for example, a derivative having a structure that contains —CO—NH— and —CO—OR (wherein R is an alkyl group and so forth)). The H in the —CO—NH— and the OH in the —CO—OH in a polyamic acid undergo dehydration by heating and so forth to produce a polyimide having a cyclic chemical structure (—CO—(N—)—CO—).

[Step (b)]

The resulting polyamic acid is then imidized by carrying out a dehydrative cyclization (i.e. a dehydrative ring closure). The procedure for this can be exemplified by procedures that employ a dehydrating agent (i.e. chemical imidization) and procedures that employ heating (i.e. thermal imidization) to 160 to 350° C. (generally treatment at 160 to 220° C. for a solution and at least 300° C. for a cast film).

The dehydrating agent used in chemical imidization can be exemplified by acid anhydrides such as acetic anhydride, propionic anhydride, benzoic anhydride and so forth, or the corresponding acid chlorides; and carbodiimide compounds such as dicyclohexylcarbodiimide and so forth. Heating at a temperature of 60 to 120° C. is preferably carried out when chemical imidization is employed.

Thermal imidization is preferably performed while removing the water produced by the dehydration reaction from the system. In this case, the water can be azeotropically removed using, for example, benzene, toluene, xylene, and so forth.

In addition, a basic catalyst can be used in the imidization if needed. Examples of the basic catalyst include pyridine, isoquinoline, trimethylamine, triethylamine, N,N-dimethylaminopyridine, imidazole, 1-methylpiperidine, 1-methylpiperazine and so forth. The dehydrating agent and the basic catalyst are each preferably used in the range of 0.1 to 8 moles per 1 mole of the acyl compound.

Chemical imidization is preferred for the imidization procedure because, for example, it enables imidization to be performed by heating at lower temperatures.

The imidization is performed so as to achieve the imidization of at least a portion, preferably at least 75 mol %, more preferably at least 80 mol %, and particularly preferably at least 85 mol % of the polyamic acid repeating units.

The resulting solution including an organic solvent and a polyamic acid and/or polyimide can be directly used as is, or can be used in a way where the polyimide and so forth is isolated from the resulting solution as a solid fraction followed by redissolution in an organic solvent and is then used. The organic solvent used for redissolution can be exemplified by those organic solvents already cited above. As an example of the procedure for isolating the polyimide and so forth, the polyimide and so forth can be precipitated by introducing the solution including the organic solvent and the polyimide and so forth into a poor solvent for polyimide such as methanol, isopropyl ether and so forth to produce a precipitate of polyimide and so forth, and separating the polyimide and so forth as a solid fraction by, for example, filtration, washing, drying and so forth. This procedure also permits removal of the dehydration catalyst (i.e. imidization catalyst) used for imidization.

In the present invention, the proportion of polyimide in the 100 mol % corresponding to the sum of the polyamic acid and polyimide, is preferably at least 75 mol % more preferably at least 80 mol %, and particularly preferably at least 85 mol %. When the polyimide proportion is less than 75 mol %, the film may exhibit a high water absorptivity and its durability may be reduced.

The resulting polyamic acid and/or polyimide has a weight-average molecular weight, expressed on a polystyrene basis, of preferably 40,000 to 500,000 and more preferably 50,000 to 400,000.

[Step (c)]

Step (c) is a step of forming a coating layer by coating a supporting substrate with the solution including an organic solvent and a polyamic acid and/or polyimide.

This supporting substrate can be exemplified by polyethylene terephthalate (PET) film, SUS sheet, and so forth.

Roll coating, gravure coating, spin coating, procedures that use a doctor blade, and so forth, can be used as the procedure for coating the supporting substrate with the solution including an organic solvent and a polyimide and so forth.

The thickness of the coating layer is not particularly limited, but can be exemplified by 1 to 250 μm.

[Step (d)]

Step (d) is a step of obtaining a film by removing the organic solvent from the coating layer by evaporation.

In specific terms, the organic solvent in the coating layer is evaporated and removed by heating the coating layer.

The heating conditions here should result in the evaporation of the organic solvent, but are not otherwise particularly limited, and can be exemplified by 1 to 5 hours at 60 to 250° C. Heating may also be carried out in two stages. For example, heating for 30 minutes at 100° C. may be followed by heating for 1 hour at 150° C. Drying in a nitrogen atmosphere or under reduced pressure may be performed if needed.

Since this step is directed simply to removing the organic solvent and is free of any requirement of carrying out imidization, the film can be obtained at lower temperatures than in the prior art. As a consequence, even when another component forming an optical element has a low heat resistance, film formation can be carried out by directly coating this component with the previously described solution containing an organic solvent and a polyimide and so forth and then evaporatively removing the organic solvent.

The obtained film can be peeled from the supporting substrate for use or can be used as is without peeling.

The main constituent of the film of the present invention is the polyimide and so forth yielded by the reaction of components (A) and (B).

The polyamic acid yielded by the reaction of components (A) and (B), for example, has at least one of the eight repeating units given by the following formulas (4-1) to (7-2).

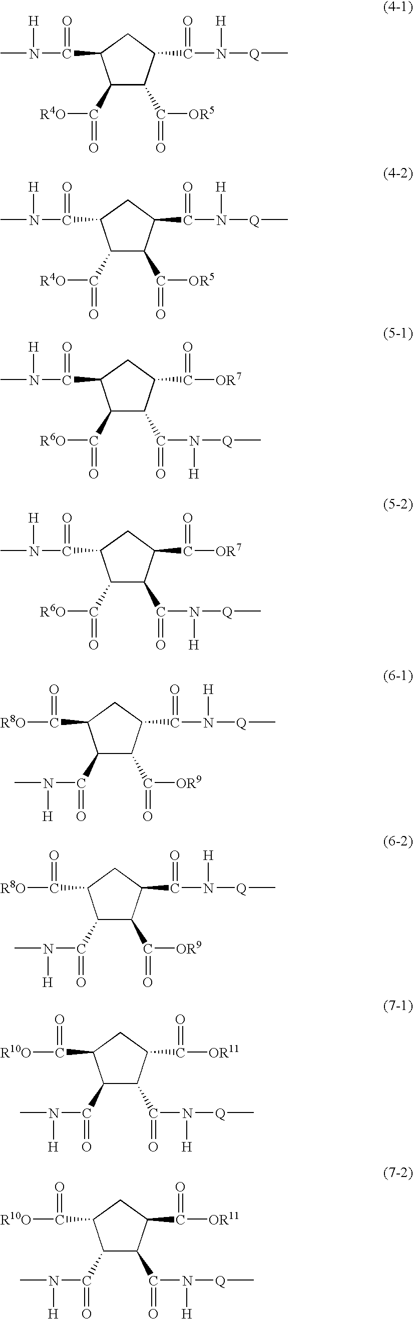

(In the preceding formulas, R¹ to R¹¹ each independently represent the hydrogen atom or an alkyl group, and Q represents a divalent organic group.)

In addition, the polyimide yielded by the reaction of components (A) and (B), for example, has a repeating unit represented by the following formula (8-1) or (8-2).

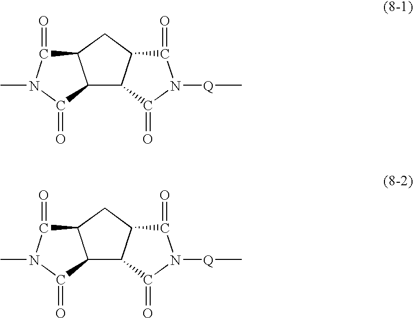

(Q in the preceding formulas is the same as the Q in the previously described formulas (4-1) to (7-2)).

The proportion of polyimide in the 100 mol % corresponding to the sum of the polyamic acid and polyimide in the film of the present invention is at least 75 mol %, preferably at least 80 mol %, and particularly preferably at least 85 mol %. When the polyimide proportion is less than 75 mol %, the film may exhibit a high water absorptivity and its durability may be reduced.

The film of the present invention has a thickness of 1 to 250 μm and preferably 5 to 200 μm. A thickness of 10 to 150 μm is particularly preferred when the film of the present invention is used as a substrate.

The glass-transition temperature (Tg) of the film of the present invention is preferably not less than 200° C. and more preferably is not less than 250° C. An excellent heat resistance can be obtained by having such a glass-transition temperature.

The film of the present invention can be used for the surrounding materials for light-emitting diodes, the surrounding materials in solar cells, the surrounding materials in flat displays, and the surrounding materials for electronic circuits. It can specifically be used for optical elements such as heat-resistant transparent films, electroconductive transparent films, and so forth. In the case of the surrounding materials for electronic circuits, the film of the present invention can also be used as a printing wiring substrate such as a flexible printed wiring substrate, a rigid printed wiring substrate, a substrate for optoelectronic printed wiring, a substrate for chip-on-film (COF), and a substrate for tape automated bonding (TAB). In the case of use as a printed wiring substrate, for example, a copper layer may also be provided for wiring. The method for providing the copper layer on the film of the present invention can be exemplified by lamination, metallization, and so forth. In the case of lamination, for example, a printed wiring substrate provided with a copper layer can be produced by hot pressing a copper foil on the film of the present invention. In the case of metallization, for example, after performing surface modification in order to cause the film of the present invention to exhibit affinity for metals, a Ni-based metal layer bonded to the polyimide and the seed layer required for wet electroplating are formed by vapor deposition or sputtering. A printed wiring substrate provided with a copper layer can be produced by applying a copper layer having a prescribed thickness by a wet plating method.

The polyimide-type solution which is provided by steps (a) and (b) and which contains organic solvent and polyimide and so forth can also be used as a polyimide-type resin composition for the surrounding materials for light-emitting diodes, the surrounding materials for solar cells, the surrounding materials for flat displays, and the surrounding materials for electronic circuits. In specific terms, it can be used as a sealant, a lens material, a material for forming a printed wiring substrate, and so forth. For example, in the case of use as a material for forming a printed wiring substrate, a printed wiring substrate can be produced by a casting procedure. Specifically, a printed wiring substrate provided with a copper layer can be produced by coating copper foil with the aforementioned polyimide-type resin composition and then heating.

The aforementioned polyimide-type resin composition can contain an organic solvent having a boiling point not greater than 150° C. as a cosolvent. This organic solvent can be exemplified by methanol, ethanol, isopropanol, tetrahydrofuran, 1,3-dioxane, 1,4-dioxane, and so forth.

A single one or a mixture of two or more of these solvents may be used.

The concentration of the polyamic acid and/or polyimide in the total mass of the polyimide-type resin composition is preferably 5 to 30 mass %.

### Examples

The present invention is specifically described in the following by Examples.

### [(A) Synthesis of Acyl Compounds]

The acyl compounds (i.e. the tetracarboxylic acid and anhydride) were prepared by the following methods, and their structures were confirmed by NMR, x-ray structural analysis, and so forth.

### Example 1

### Synthesis of a Tetracarboxylic Acid

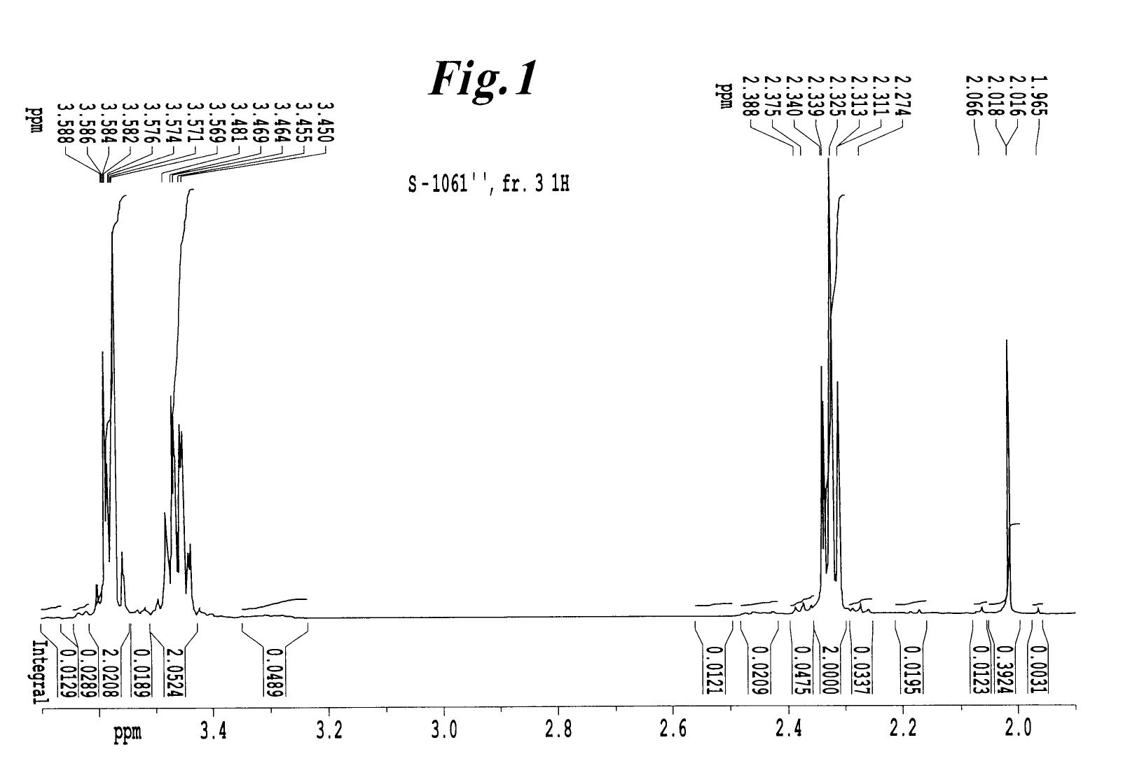

12.31 g (50.0 mmol) of all-cis 1,2,3,4-cyclopentanetetracarboxylic acid was heated for 5 hours in a oven at 285° C. under a nitrogen atmosphere. After cooling to room temperature, 100 mL of pure water was added and stirring was carried out for 2 hours until the solid had dissolved. Active carbon was added and filtration was performed to obtain a colorless and transparent liquid. This liquid was evaporated to dryness to obtain crude 1-cis-2-cis-3-trans-4-trans-cyclopentanetetracarboxylic acid and various tetracarboxylic acid isomers (yield=10.75 g). Structural analysis of this 1-cis-2-cis-3-trans-4-trans-cyclopentanetetracarboxylic acid was performed by NMR (¹H, D.O) (see FIG. 1).

### Example 2

### Synthesis of an Acid Anhydride

150 mL of acetic anhydride was added to 10.75 g (43.6 mmol) of the crude tetracarboxylic acid obtained in Example 1, and stirring was carried out for 3 hours at 100° C. Active carbon was then added, and stirring was carried out for 20 minutes. After that, hot filtration was performed. The filtrate was cooled in an ice bath, and the precipitate was recovered by filtration. The precipitate was washed several times with acetic anhydride to yield colorless crystals of 1-cis-2-cis-3-trans-4-trans-cyclopentanetetracarboxylic acid dianhydride (yield=4.85 g, percent yield=52.9 mass).

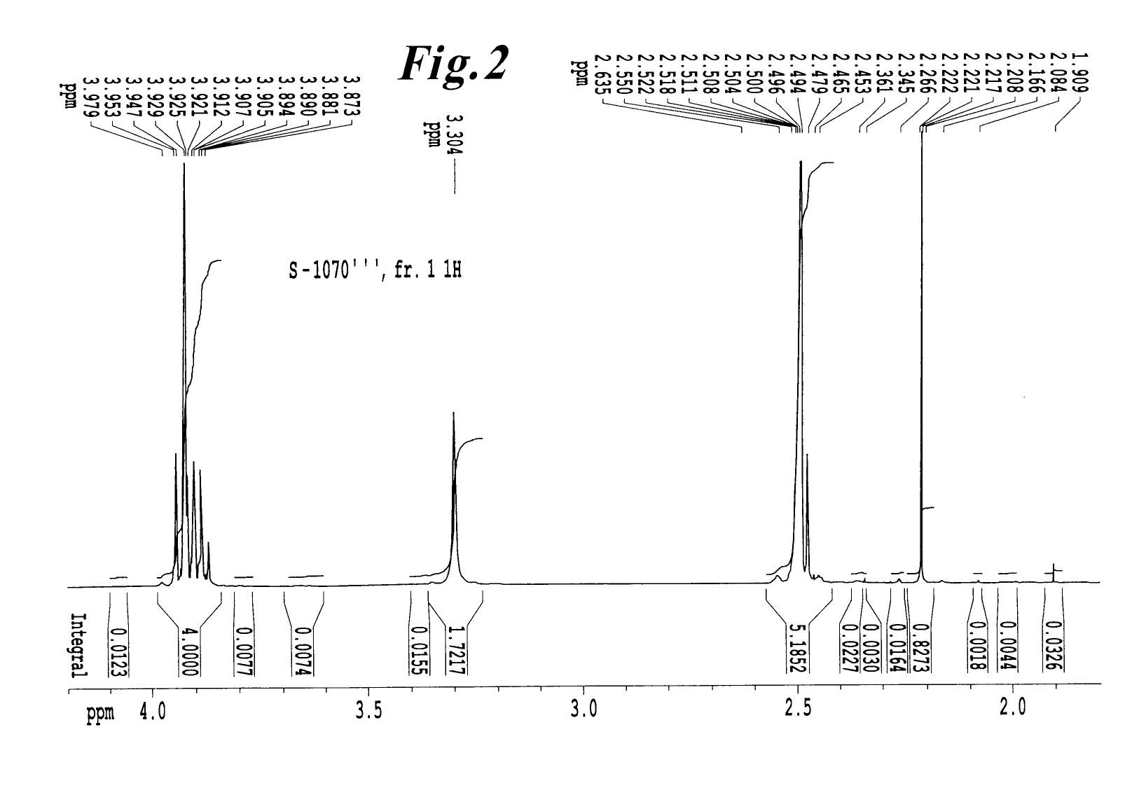

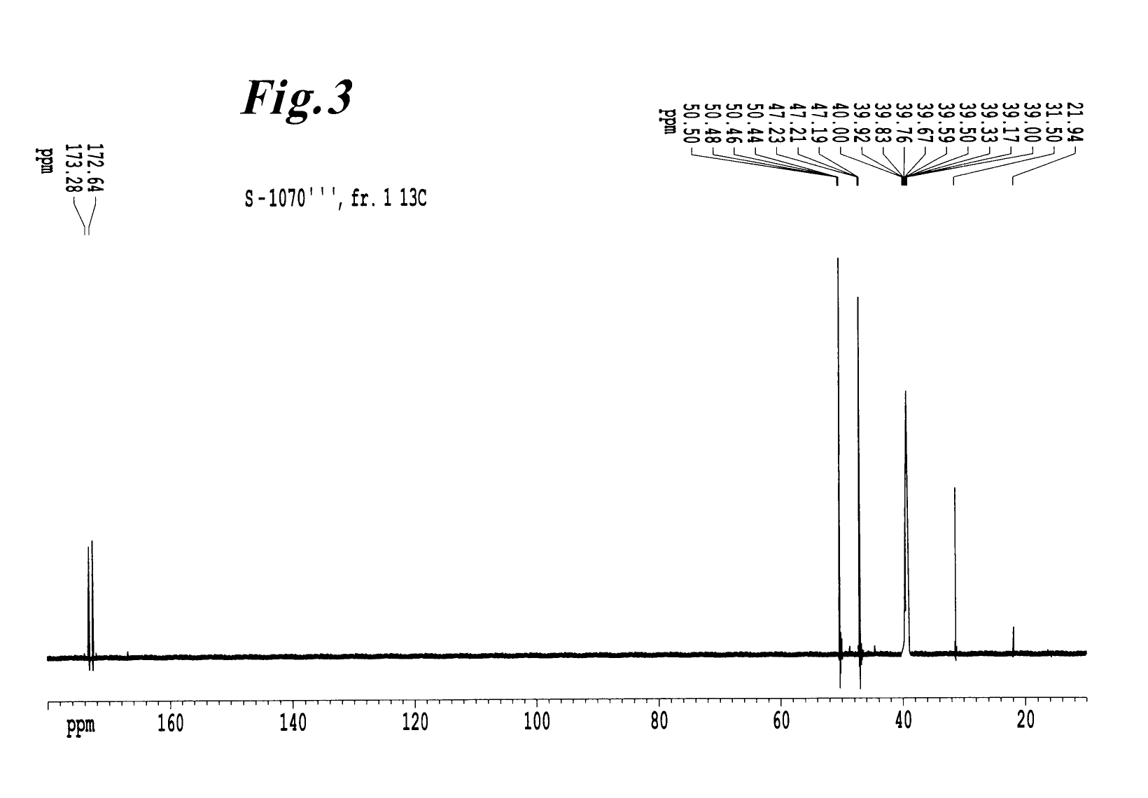

Structural analysis of this tetracarboxylic acid anhydride was performed by NMR (H, DMSO-d₆; C, CDCl) (see FIGS. 2 and 3). FIG. 2 gives the results of ¹H NMR in DMSO-d₆. FIG. 3 gives the results of ¹³C NMR in CDCl.

### [X-Ray Structural Analysis]

1.30 g of methylamine (40% aqueous solution, 15.0 mmol) was added to a 100 mL four-neck flask equipped with a thermometer, stirrer, nitrogen inlet tube, Dean-Stark trap, and condenser. Then, after replacing the interior atmosphere of the flask by nitrogen, 10 mL of xylene was added and stirring was performed to homogeneity. 0.15 g (0.5 mmol) of the 1-cis-2-cis-3-trans-4-trans-cyclopentanetetracarboxylic acid dianhydride obtained in Example 2 was added at room temperature to the resulting solution. Heating to 150° C. was then carried out and stirring was continued for 4 hours, thus yielding a bisimide powder (yield=0.114 g, percent yield=96.2 mass %). The obtained powder was purified by sublimation.

This bisimide powder was submitted to structural analysis by x-ray structural analysis. The results are given in Table 1. The results of the structural analysis demonstrated that the tetracarboxylic acid obtained in Synthesis Example 1 was 1-cis-2-cis-3-trans-4-trans-cyclopentanetetracarboxylic acid and that the acid anhydride obtained in Synthesis Example 2 was 1-cis-2-cis-3-trans-4-trans-cyclopentanetetracarboxylic acid dianhydride.

TABLE 1
Crystal data

| | |
|---|---|
| space group | P2/n |
| crystal system | monoclinic |
| lattice constants (A) | a = 19.35000 |
| | b = 24.9339 |
| | c = 6.6607 |
| | β = 96.841° |
| volume of unit cell (A³) | V = 3190.7 |
| number of atoms in the unit | Z = 12 |

Measuring method

| | |
|---|---|
| x-ray generator | RU-H2R made by Rigaku Corp. |
| x-ray source | CuKα radiation |
| goniometer | Model AFC-7R made by |
| | Rigaku Corp. |
| collimator | 1.0 mm φ |
| detector | scintillation counter |
| attenuator | Ni foil (factor = 9.03) |
| receiving slit | horizontal slit 3.0 mm |
| | vertical slit 3.0 mm |
| scan method | ω-2θ scan |
| scan rate | 16.0°/min (in omega) |
| maximum no. of repeat scans | 3 times (I < 10.0σ (I)) |
| scan width | (1.37 + 0.30 tanθ)° |
| 2θₘₐₓ | 149.7° |
| number of independent reflections | 6448 (Rᵢₙₜ = 0.0015) |
| absorption correction | correction by φ scan |
| measurement temperature | 19.0 ± 2.5° C. |

Structure analysis and refinement methods

| | |
|---|---|
| phase determination method | direct method (SHELXS-97) |
| structure refinement method | full-matrix least-squares |
| | method (SHELXS-97) |
| structure factor for refinement | F² |
| temperature factor | anisotropic (nonhydrogen atom) |
| | isotropic (hydrogen atom) |
| number of reflections used for | 6448 (all the independent |
| refinement | reflections) |
| correction of extinction effect | no correction |
| R factor | R1(F) = 0.037 (I > 2σ (I)) |
| | wR(F²) = 0.016 |

### [(B) Film Production]

### Example 3

### Production of Film of the Present Invention

9.92 g (24.2 mmol) of 2,2-bis[4-(4-aminophenoxy)phenyl]propane was added to a 300 mL four-neck flask equipped with a thermometer, stirrer, nitrogen inlet tube, and condenser. After that, after replacing the interior atmosphere of the flask by nitrogen, 58 mL of N-methyl-2-pyrrolidone (hereinafter referred to as NMP) was added, and stirring was carried out to homogeneity. 5.08 g (24.2 mmol) of the 1-cis-2-cis-3-trans-4-trans-cyclopentanetetracarboxylic acid dianhydride obtained in Example 2 was added at room temperature to the resulting solution, and stirring was continued for 24 hours at the same temperature to obtain a polyamic acid solution.

2.5 mL of N-methylpiperidine and 6.8 mL acetic anhydride were added to the resulting polyamic acid solution, and imidization was performed by stirring for 4 hours at 75° C. After cooling to room temperature, introduction into a large volume of methanol was performed, and a polymer was then isolated by filtration. The obtained polymer was vacuum dried overnight at 60° C. to yield a white powder (yield=13.4 g, percent yield=94.6 mass %).

The resulting polymer was redissolved in N,N-dimethylacetamide (DMAc) to prepare the 20 mass % of resin solution. A substrate of polyethylene terephthalate (PET) was coated with this resin solution using a doctor blade (100 μm gap). A film was made by drying this PET substrate at 100° C. for 30 minutes and then 150° C. for 60 minutes. The film was then subsequently peeled away from the PET substrate. After that, the film was further dried under reduced pressure for 3 hours at 150° C. to obtain a film having a thickness of 20 μm.

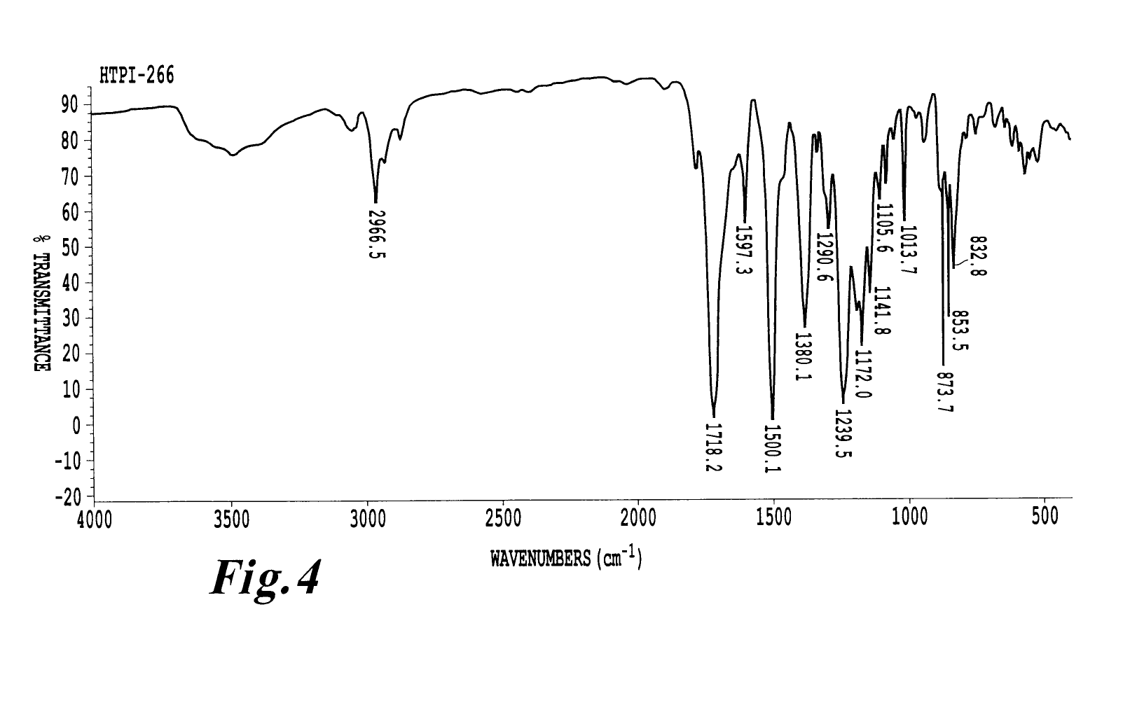

Structural analysis was performed on the aforementioned polymer by IR (KBr procedure). According to the results, the characteristic absorptions of the carbonyl group were observed at 1781 cm⁻¹ and 1718 cm⁻¹ (see FIG. 4).

In addition, the polymer was submitted to an evaluation of the weight-average molecular weight, imidization rate, imide group concentration (theoretical value assuming that the imidization rate was 100 mol %), solubility in organic solvent, and glass-transition temperature according to the methods described below. The weight-average molecular weight was measured on the polyimide and polyamic acid before and after imidization.

The results are given in Table 2.

### (1) Weight-Average Molecular Weight

The weight-average molecular weight was measured using a Model HLC-8020 GPC instrument made by Tosoh Corporation. N-methyl-2-pyrrolidone (NMP) containing lithium bromide and phosphoric acid was used as the solvent, and the molecular weight, expressed on a polystyrene basis, was determined at a measurement temperature of 40° C.

### (2) Imidization Rate (Cyclization Rate)

The cyclization rate of the polyimide was measured using ¹H-NMR. d-DMSO was used for the solvent. The cyclization rate was determined from the ratio between the integrated peak value for the N—H proton on the uncyclized amide group and the integrated peak value for the —CH— (methylene) protons originating from the alicyclic diamine or the integrated peak value for the aromatic protons originating from the aromatic diamine.

### (3) Solubility in Organic Solvent

The polymer was dissolved in N,N-dimethylacetamide and adjusted to give the 20 mass % solution, and the solubility at room temperature was evaluated. Complete dissolution is indicated with an open circle, while a swollen or insoluble polymer is indicated with an “×”.

### (4) Glass-Transition Temperature (Tg)

The glass-transition temperature of the obtained polymer or film was measured using a Model 8230 DSC measurement instrument made by Rigaku Corporation at a rate of temperature rise of 20° C./minute.

### (5) Modulus, Rupture Strength, and Elongation

The film was punched by a #7 dumbbell and the elongation at rupture was measured using a Model 5543 made by Instron Corporation at a pull rate of 500 mm/minute. The measurement was performed at 23° C. and 50% RH.

### Example 4

### Production of Film of the Present Invention

7.50 g (35.7 mmol) of 4,4′-diaminodicyclohexylmethane was added to a 300 mL four-neck flask equipped with a thermometer, stirrer, nitrogen inlet tube, and condenser. After that, after replacing the interior atmosphere of the flask by nitrogen, 58 mL of N-methyl-2-pyrrolidone (NMP) was added, and stirring was carried out to homogeneity. 7.50 g (35.7 mmol) of 1-cis-2-cis-3-trans-4-trans-cyclopentanetetracarboxylic acid dianhydride was added at room temperature to the resulting solution, and after the addition the precipitated salt was dissolved by stirring for 15 minutes at 120° C. Stirring was then continued for 24 hours at this same temperature to produce a polyamic acid solution.

2.5 mL of N-methylpiperidine and 6.8 mL of acetic anhydride were added to the resulting polyamic acid solution, and imidization was performed by stirring for 3 hours at 75° C. After cooling to room temperature, introduction into a large volume of methanol was performed, and a polymer was then isolated by filtration. The obtained polymer was vacuum dried overnight at 60° C. to yield a white powder (yield=12.8 g, percent yield=93.5 mass).

A film having a thickness of 20 μm was then obtained by processing the obtained polymer in the same way as that in Example 3.

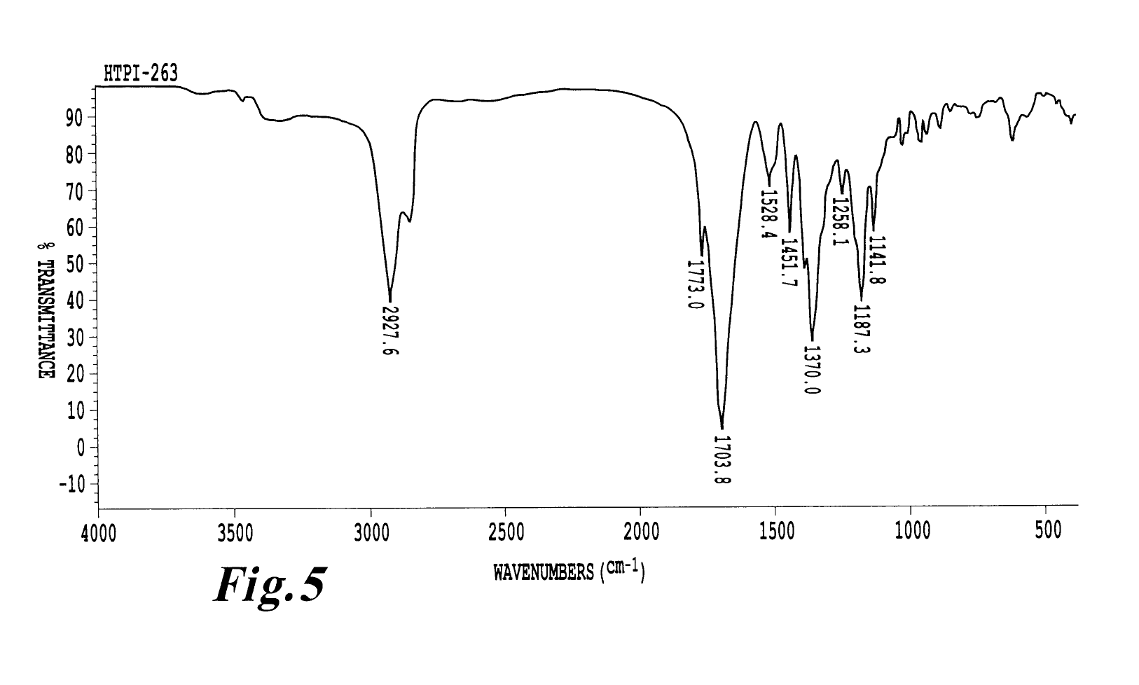

Structural analysis of the obtained polymer was performed in the same way as that in Example 3. According to the results, the characteristic absorptions of the carbonyl group were observed at 1773 cm⁻¹ and 1704 cm⁻¹ (see FIG. 5).

The various properties of the obtained polymer were also evaluated in the same way as that in Example 3. The results are shown in Table 2.

### Comparative Example 1

### Production of Film for Comparison

The procedure in the same way as that of Example 3 was performed with the following exceptions: 5.08 g (24.2 mmol) of all-cis cyclopentanetetracarboxylic acid dianhydride (i.e., 1-cis-2-cis-3-cis-4-cis-cyclopentanetetracarboxylic acid dianhydride) was used in place of the 1-cis-2-cis-3-trans-4-trans-cyclopentanetetracarboxylic acid dianhydride, and the amounts of addition of the 2,2-bis[4-(4-aminophenoxy)phenyl]propane, N-methylpiperidine, and acetic anhydride were changed, respectively, to 9.92 g (24.2 mmol), 3.5 mL, and 9.8 mL. A polymer in the form of a white powder was obtained (yield=12.9 g, percent yield=91.3 mass %).

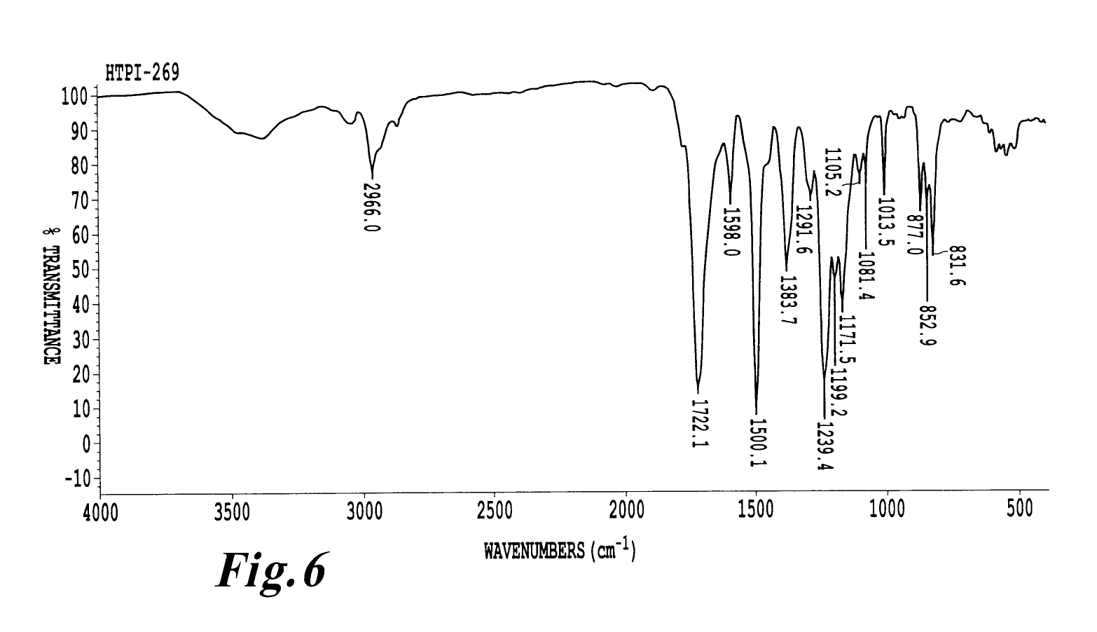

Structural analysis of the obtained polymer was performed in the same way as that in Example 3. According to the results, the characteristic absorptions of the carbonyl group were observed at 1779 cm⁻¹ and 1722 cm⁻¹ (see FIG. 6). The results are shown in FIG. 2.

The various properties of the obtained polymer were also evaluated in the same way as those in Example 3. The results are shown in Table 2. In this case, a film could not be produced due to the low molecular weight of the obtained polymer.

### Comparative Example 2

### Production of Film for Comparison

The procedure in the same way as that of Example 4 was performed with the following exceptions: 7.50 g (35.7 mmol) of all-cis cyclopentanetetracarboxylic acid dianhydride was used in place of the 1-cis-2-cis-3-trans-4-trans-cyclopentanetetracarboxylic acid dianhydride, and the amounts of addition of the 4,4′-diaminodicyclohexylmethane, N-methylpiperidine, and acetic anhydride were changed, respectively, to 7.50 g (35.7 mmol), 3.5 mL, and 10.4 mL. A polymer in the form of a white powder was obtained (yield=12.6 g, percent yield=92.2 mass %).

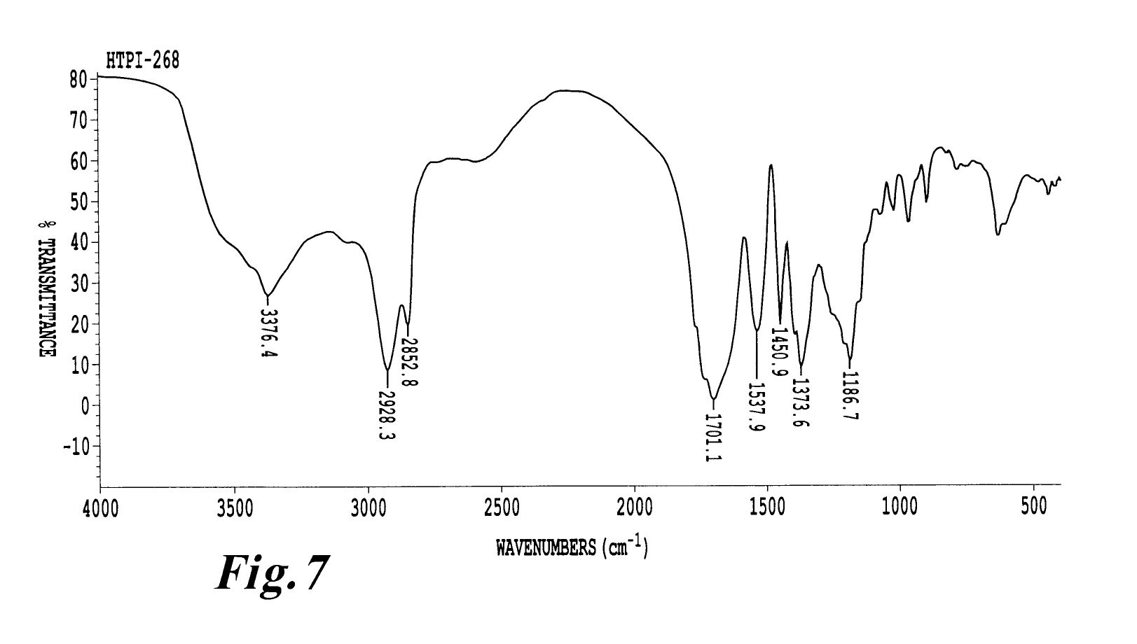

Structural analysis of the obtained polymer was performed in the same way as that in Example 3. According to the results, the characteristic absorptions of the carbonyl group were observed at 1770 cm⁻¹ and 1702 cm⁻¹ (see FIG. 7).

The various properties of the obtained polymer were also evaluated in the same way as those in Example 3. The results are shown in Table 2. In this case, a film could not be produced due to the low molecular weight of the obtained polymer.

### Comparative Example 3

### Production of Film for Comparison

The procedure in the same way as that of Example 4 was followed with the following exceptions: 7.74 g (34.5 mmol) of 1,2,4,5-cyclohexanetetracarboxylic acid dianhydride was used in place of the 1-cis-2-cis-3-trans-4-trans-cyclopentanetetracarboxylic acid dianhydride, and the amounts of addition of the 4,4′-diaminodicyclohexylmethane, N-methylpiperidine, and acetic anhydride were changed, respectively, to 7.26 g (34.5 mmol), 3.5 mL, and 10.4 mL. A polymer in the form of a white powder was obtained (yield=13.1 g, percent yield=95.4 mass %).

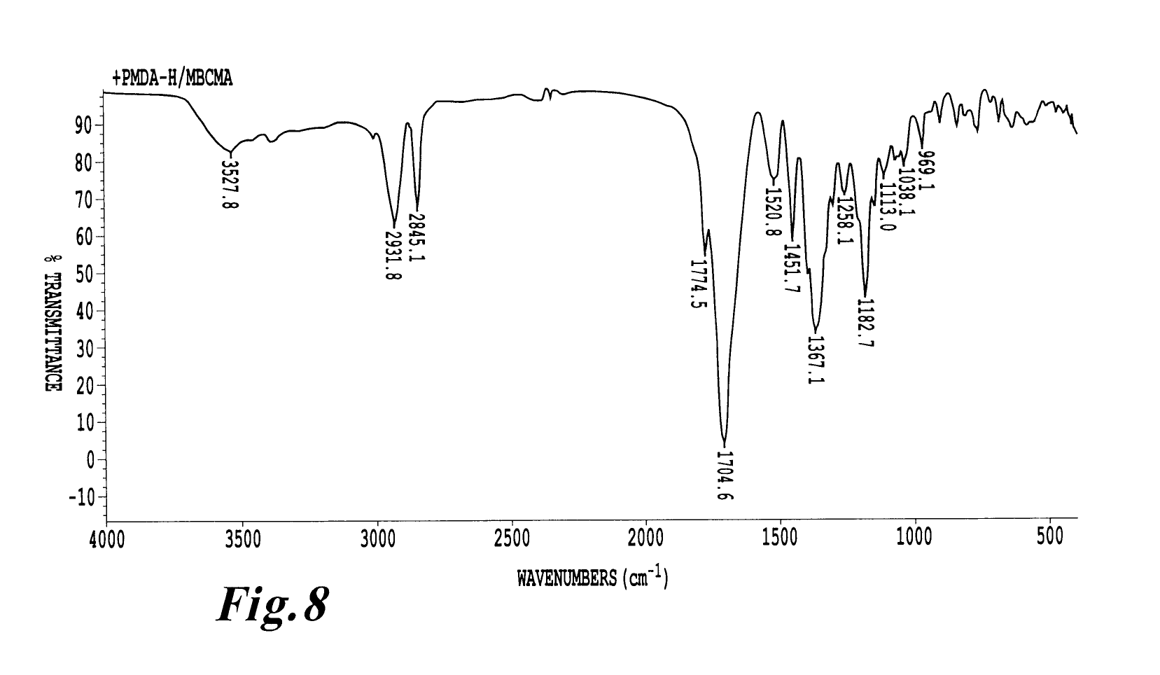

Structural analysis of the obtained polymer was performed in the same way as that in Example 3. According to the results, the characteristic absorptions of the carbonyl group were observed at 1775 cm⁻¹ and 1705 cm⁻¹ (see FIG. 8).

The various properties of the obtained polymer were also evaluated in the same way as those in Example 3. The results are shown in Table 2.

### Comparative Example 4

### Production of Film for Comparison

7.76 g (36.9 mmol) of 4,4′-diaminodicyclohexylmethane was added to a 300 mL four-neck flask equipped with a thermometer, stirrer, nitrogen inlet tube, Dean-Stark trap, and condenser. After that, after replacing the interior atmosphere of the flask by nitrogen, 58 mL of NMP was added, and stirring was carried out to homogeneity. 7.24 g (36.9 mmol) of cyclobutanetetracarboxylic acid dianhydride was added at room temperature to the resulting solution, and after the addition the precipitated salt was dissolved by stirring for 15 minutes at 120° C. Stirring was then continued for 24 hours at the same temperature to produce a polyamic acid solution.

A substrate of polyethylene terephthalate (PET) was then coated with the resulting polyamic acid solution using a doctor blade (100 μm gap). A film was made by drying the polyamic acid solution at 100° C. for 30 minutes and then 120° C. for 60 minutes. The film was subsequently peeled away from the PET substrate. After that, the film was further dried under reduced pressure for 2 hours at 250° C. to obtain a film having a thickness of 21 μm.

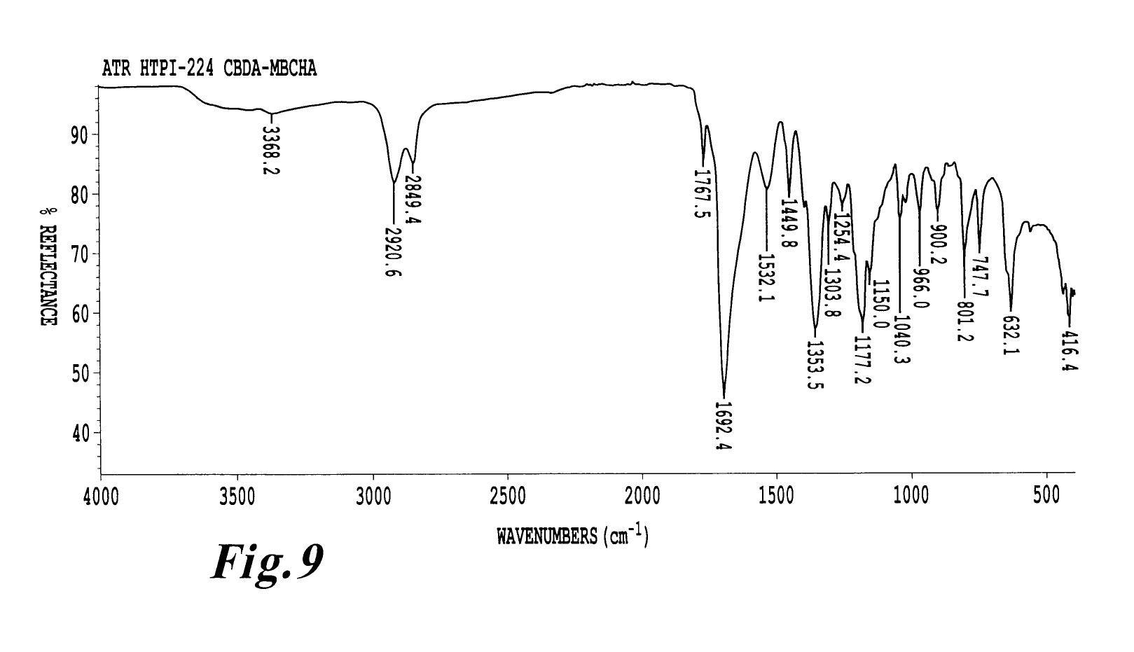

Structural analysis of the obtained polymer was performed in the same way as that in Example 3. According to the results, the characteristic absorptions of the carbonyl group were observed at 1768 cm⁻¹ and 1692 cm⁻¹ (see FIG. 9).

The various properties of the obtained polymer (polyamic acid) were also evaluated in the same way as those in Example 3. The results are shown in Table 2.

TABLE 2

| | | | | | | | | Rupture | |
|---|---|---|---|---|---|---|---|---|---|
| | component | component | | imidization | | | modulus | strength | elongation |
| | (A) | (B) | Mw | rate (%) | solubility | Tg(° C.) | (Gpa) | (Mpa) | (%) |
| Example 3 | t-CPDA¹⁾ | BAPP⁵⁾ | 1.5E+05 | 95 | ∘ | 264 | 2.9 | 82 | 18 |
| Example 4 | t-CPDA | MBCHA⁶⁾ | 1.2E+05 | 89 | ∘ | >350 | 2.7 | 96 | 48 |
| Comparative | c-CPDA²⁾ | BAPP | 4.7E+03 | 83 | ∘ | 212 | — | — | — |
| Example 1 | | | | | | | | | |
| Comparative | c-CPDA | MBCHA | 4.6E+03 | 69 | ∘ | 262 | — | — | — |
| Example 2 | | | | | | | | | |
| Comparative | PMDAH³⁾ | MBCHA | 8.7E+04 | 93 | ∘ | 265 | 2.3 | 73 | 12 |
| Example 3 | | | | | | | | | |
| Comparative | CBDA⁴⁾ | MBCHA | 5.3E+04 | — | x | >350 | 3 | 101 | 9 |
| Example 4 | | | | | | | | | |

¹⁾t-CPDA: 1-cis-2-cis-3-trans-4-trans-cyclopentanetetracarboxylic acid dianhydride
²⁾c-CPDA: all-cis cyclopentanetetracarboxylic acid dianhydride
³⁾PMDAH: 1,2,4,5-cyclohexanetetracarboxylic acid dianhydride
⁴⁾CBDA: cyclobutanetetracarboxylic acid dianhydride
⁵⁾BAPP: 2,2-bis[4-(4-aminophenoxy)phenyl]propane
⁶⁾MBCHA: 4,4′-diaminodicyclohexylmethane

Table 2 demonstrates that the polyimide-type materials prepared using a novel acyl compound of the present invention (Examples 3 and 4) provide an excellent workability in molding into the film form due to their high solubility in organic solvent, and provide films that exhibit an excellent heat resistance due to their high glass-transition temperature. On the other hand, in the case of Comparative Examples 1 and 2, which employed an acyl compound outside the scope of the present invention, the glass-transition temperatures (i.e. heat resistance) of these Comparative Examples are lower than those in Examples 3 and 4 which employed the same component (B) as in these Comparative Examples, and the weight-average molecular weight of these Comparative Examples are lower than those in Examples 3 and 4. In the case of Comparative Example 3, the glass-transition temperature (i.e. heat resistance) of Comparative Example is also shown to be lower than that in Example 4 which employed the same component (B) as in Comparative Example 3. In the case of Comparative Example 4, in which imidization of the polyamic acid was not carried out, it is shown that the solubility in organic solvent is low and the workability in film formation is poor.

What is claimed is:

1. A polyimide-type material comprising at least one selected from polyamic acids and polyimides obtained by reacting
(A) at least one acyl compound selected from the group consisting of 1-cis-2-cis-3-trans-4-trans-cyclopentanetetracarboxylic acid, 1-cis-2-cis-3-trans-4-trans-cyclopentanetetracarboxylic acid dianhydride, and reactive derivatives thereof, with
(B) an imino-forming compound.

2. The polyimide-type material according to claim 1, wherein the imino-forming compound (B) is a diamine compound.

3. A polyimide-type resin composition comprising the polyimide-type material according to claim 1 or 2 and an organic solvent.

4. A film comprising the polyimide-type material according to claim 1 or 2.

5. The film according to claim 4, used for an optical element.

6. The film according to claim 4, used for a printed wiring substrate.

7. A method of producing the film according to any one of claims 4 to 6, comprising the steps of:
forming a coating layer by coating a substrate with a solution comprising organic solvent and a polyamic acid and/or polyimide obtained by reacting the acyl compound (A) with the imino-forming compound (B); and
obtaining a film by removing the solvent from the coating layer by evaporation.

8. 1-cis-2-cis-3-trans-4-trans-cyclopentanetetracarboxylic acid.

9. 1-cis-2-cis-3-trans-4-trans-cyclopentanetetracarboxylic acid dianhydride.

10. A material for synthesizing a polyamic acid and/or a polyimide, comprising 1-cis-2-cis-3-trans-4-trans-cyclopentanetetracarboxylic acid and/or 1-cis-2-cis-3-trans-4-trans-cyclopentanetetracarboxylic acid dianhydride.

11. A polyamic acid obtained by reacting
(A) at least one acyl compound selected from the group consisting of 1-cis-2-cis-3-trans-4-trans-cyclopentanetetracarboxylic acid, 1-cis-2-cis-3-trans-4-trans-cyclopentanetetracarboxylic acid dianhydride, and reactive derivatives thereof, with
(B) an imino-forming compound.

12. A polyimide obtained by reacting
(A) at least one acyl compound selected from the group consisting of 1-cis-2-cis-3-trans-4-trans-cyclopentanetetracarboxylic acid, 1-cis-2-cis-3-trans-4-trans-cyclopentanetetracarboxylic acid dianhydride, and reactive derivatives thereof, with
(B) an imino-forming compound.
